(19) United States
(12) Patent Application Publication
(10) Pub. No.: US 2005/0101922 A1
(43) Pub. Date: May 12, 2005

(54) Suction canister and drainage of same

(75) Inventors: Barry G. Anderson, Sheboygan, WI (US); Joseph M. Hand, Sheboygan Falls, WI (US)

Correspondence Address: MICHAEL BEST & FRIEDRICH, LLP, 100 E WISCONSIN AVENUE, MILWAUKEE, WI, 53202, US

(73) Assignee: Bemis Manufacturing Company, Sheboygan Falls, WI (US)

(21) Appl. No.: 10/703,340

(22) Filed: Nov. 7, 2003

(51) Int. Cl. A61M 1/00

(52) U.S. Cl. 604/317

(57) ABSTRACT

A suction canister including a rigid walled fluid container having an interior, a cover secured to the container, the cover having therein a port, a first port wall extending into the interior, and a second port wall extending in a direction opposite the interior, and a conduit coupled to the first port wall and extending into the interior.

### BACKGROUND OF THE INVENTION

The invention relates to hard canister type medical suction apparatus.

Medical suction apparatus are used in hospital environments and particularly during various surgical procedures to drain and store fluid from a patient. In general, medical suction apparatus are used in conjunction with a vacuum source that enables the fluid to be drained from the patient. One type of medical suction apparatus used to drain and contain fluid from a patient is a suction canister. Suction canisters generally include a rigid walled receptacle for containing the fluid and a cover with a vacuum port and a patient port. A suction conduit connects the vacuum port to a vacuum source, and a patient conduit conveys fluid from the patient into the receptacle through the patient port. When the suction conduit is connected to the vacuum source, a negative pressure gradient is created in the interior of the receptacle such that fluid is drawn from the patient into the suction canister via the patient conduit.

It has become important in environments such as hospitals to eliminate the handling of and thus reduce personnel exposure to bodily fluids. Hospitals typically dispose of the fluid contained in a suction canister in various ways. Fluid can be poured from the receptacle through a port in the cover down the hospital sink and into the sewer system, can be incinerated as a liquid or solid, or can be disposed of at an approved hazardous waste site.

### SUMMARY OF THE INVENTION

The invention provides a medical apparatus including a suction canister having a rigid walled receptacle having an interior for containing fluid, a cover having a port therein, and having a first conduit coupled to the port and extending into the interior. The medical apparatus further includes a drainage device and a second conduit including a first end coupled to the drainage device and a second end coupled to the port so that fluid in the interior of the receptacle can flow through the first conduit and out of the port to the drainage device via the second conduit.

The invention also provides a medical apparatus including a suction canister having a rigid walled receptacle having an interior and having a cover, the cover having therein a port, a first fluid connector coupled to the port and extending into the interior, a drainage station, and a second fluid connector including a first end coupled to the drainage station and a second end coupled to the port. Fluid in the interior of the receptacle flows through the first fluid connector to the drainage station via the second fluid connector.

The invention also provides a suction canister including a rigid walled fluid container having an interior, a cover secured to the container, the cover having therein a port, a first port wall extending into the interior, and a second port wall extending in a direction opposite the interior. The suction canister further includes a conduit coupled to the first port wall and extending into the interior.

The invention also provides a suction canister including a rigid walled container having an interior, a cover secured to the container, the cover having therein a port, and a conduit coupled to the port and extending into the interior.

Other features and advantages of the invention will become apparent to those of ordinary skill in the art upon review of the following description, claims and drawings.

### BRIEF DESCRIPTION OF THE DRAWINGS

FIG. 1 is a perspective view of a suction canister.

FIG. 2 is a sectional view of the suction canister connected to a drainage device.

Before one embodiment of the invention is explained in detail, it is to be understood that the invention is not limited in its application to the details of construction and the arrangement of components set forth in the following description or illustrated in the drawings. The invention is capable of other embodiments and of being practiced or being carried out in various ways. Also, it is to be understood that the phraseology and terminology used herein is for the purpose of description and should not be regarded as limiting.

### DETAILED DESCRIPTION OF THE INVENTION

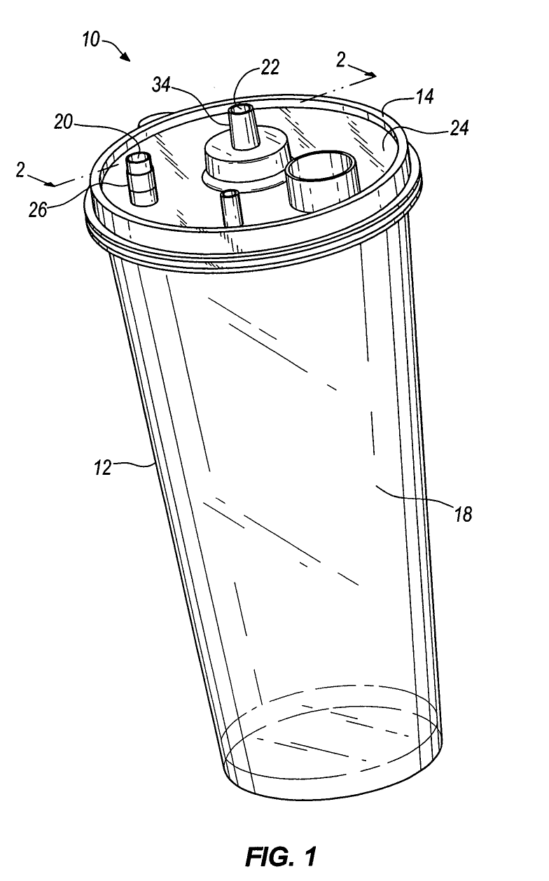

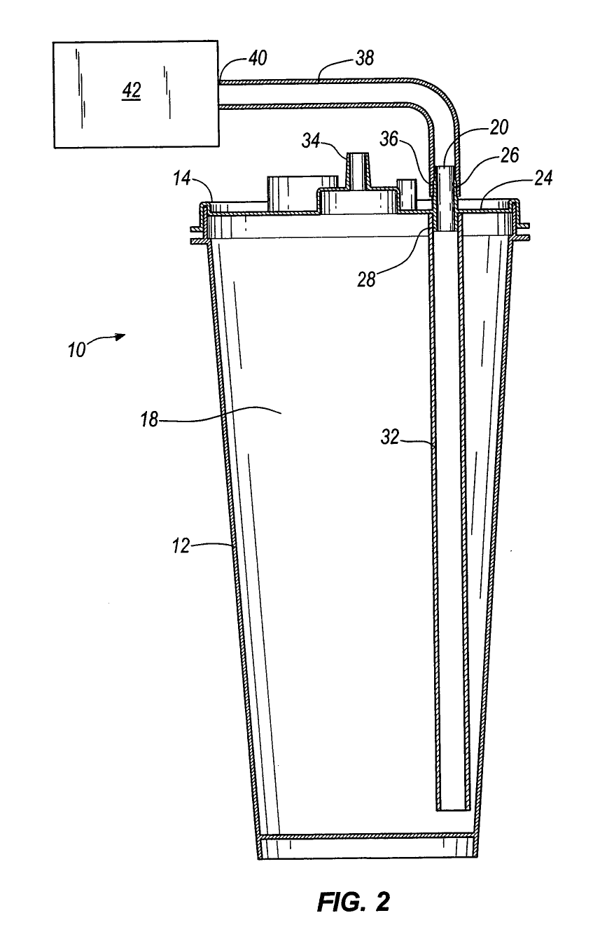

With reference to FIGS. 1 and 2, there is shown a suction canister 10 embodying the invention. The suction canister 10 includes a rigid walled receptacle or container 12 and a cover 14. The receptacle 12 is closed except for an opening at the top, with the cover 14 closing the opening. The receptacle 12 includes an interior 18 and is preferably manufactured of clear unbreakable plastic with a volume capacity that can vary as desired.

The cover 14 includes a patient port 20, a suction port 22 and other ports as needed in a top wall 24. A port wall or boss 26 extends upwardly from the patient port 20 and a port wall or boss 28 extends downwardly from the patient port 20. The walls 26 and 28 can be integral or could be separate components. A conduit or fluid connector 32 is coupled to the port wall 28 by forcing the conduit 32 over the port wall 28. After being coupled, the conduit 32 extends or hangs down into the interior 18 of the receptacle 12. Preferably, the conduit 32 extends into the interior 18 at least 75% of the length of the receptacle 12 and can also extend the length of the receptacle 12 terminating adjacent the bottom wall of the receptacle 12.

To enable communication between the patient and the patient port 20, one end of a patient conduit (not shown) is coupled to the cover 14 by forcing the patient conduit over the port wall 26. The other end of the patient conduit communicates with the patient (not shown).

A port wall 34 extends upwardly from the top wall 24. A suction conduit (not shown) is connectable to the suction port 22 to enable communication between the interior 18 and a suction source, such as a hospital suction system. To drain fluid from a patient, the patient and suction conduits are respectively secured to the patient and suction ports 20 and 22 and fluid is drained from a patient as is conventionally known.

With reference to FIG. 2, when it is desired to drain the fluid contained in the interior 18 of the suction canister 10, the suction port 22 is capped and a first end 36 of a conduit or fluid connector 38 is coupled to the port wall 26 and the second end 40 of the conduit 38 is secured to a drainage device or station 42. Fluid flows from the interior 18 of the suction canister 10, through the conduit 32, and through the conduit 38 to the drainage device 42.

It should be noted that the conduits 32 and 38 could be coupled to other ports than what is illustrated and described if it is desired to couple them to a port other than the patient port 20.

The drainage device 42 shown herein can operate using various methods to drain the suction canister 10 such as a venturi action, a pumping action, or the like. An example of a drainage device is the Eductor Fluid Management System available from Deknatel or Bemis Manufacturing Company. However, it should be noted that other drainage devices can be utilized and the invention herein is not limited to use of the Eductor Fluid Management System to drain the suction canister shown herein.

Various features and advantages of the invention are set forth in the following claims.

1. A medical apparatus comprising:
a suction canister including a rigid walled receptacle having an interior for containing fluid, a cover having a port therein, and a first conduit coupled to the port and extending into the interior;
a drainage device; and
a second conduit including a first end coupled to the drainage device and a second end coupled to the port so that fluid in the interior can flow through the first conduit and out of the port to the drainage device via the second conduit.

2. The medical apparatus of claim 1 wherein the cover includes a first port wall extending from the port in a direction away from the interior and a second port wall extending from the port in a direction toward the interior, and wherein the second end of the second conduit is coupled to the first port wall and the first conduit is coupled to the second port wall.

3. The medical apparatus of claim 2 wherein the first conduit is coupled to the second port wall via a friction fit.

4. A medical apparatus comprising:
a suction canister including a rigid walled receptacle having an interior and a cover, the cover having therein a port;
a first fluid connector coupled to the port and extending into the interior;
a drainage station; and
a second fluid connector including a first end coupled to the drainage station and a second end coupled to the port, whereby fluid in the interior flows through the first fluid connector and to the drainage station via the second fluid connector.

5. The medical apparatus of claim 4 wherein the first and second fluid connectors are flexible tubing.

6. A suction canister comprising:
a rigid walled fluid container having an interior;
a cover secured to the container, the cover having therein a port, a first port wall extending into the interior, and a second port wall extending in a direction opposite the interior; and
a conduit coupled to the first port wall and extending into the interior.

7. The suction canister of claim 6 and further including a second conduit coupled to the second port wall.

8. The suction canister of claim 6 wherein the first port wall and the second port wall are connected.

9. The suction canister of claim 6 wherein the container has a bottom wall and the conduit has a first end coupled to the first port wall and a second end positioned adjacent the bottom wall.

10. The suction canister of claim 6 wherein the container has a length and wherein the first conduit extends into the interior at least 75% of the length of the container.

11. The suction canister of claim 6 wherein the conduit is coupled to the first port wall via a friction fit.

12. A suction canister comprising:
a rigid walled container having an interior;
a cover secured to the container, the cover having therein a port; and
a conduit coupled to the port and extending into the interior.

13. The suction canister of claim 12 and further including a second conduit coupled to the port and in fluid communication with the conduit.

14. The suction canister of claim 12 wherein the conduit is flexible.

15. The suction canister of claim 12 wherein a port wall extends from the port into the interior and wherein the conduit is coupled to the port wall.

16. The suction canister of claim 15 wherein the conduit is coupled to the port wall via a friction fit.
